(19) United States
(12) Patent Application Publication
(10) Pub. No.: US 2013/0157307 A1
(43) Pub. Date: Jun. 20, 2013

(54) Process of Producing A Fermentation Product

(71) Applicants: Chee-Leong Soong, Raleigh, NC (US); Shiro Fukuyama, Chiba (JP); Allan Noergaard, Lyngby (DK); Preben Nielsen, Hoersholm (DK); Peter Rahbek Oestergaard, Virum (DK)

(72) Inventors: Chee-Leong Soong, Raleigh, NC (US); Shiro Fukuyama, Chiba (JP); Allan Noergaard, Lyngby (DK); Preben Nielsen, Hoersholm (DK); Peter Rahbek Oestergaard, Virum (DK)

(73) Assignees: NOVOZYMES NORTH AMERICA, INC., Franklinton, NC (US); NOVOZYMES A/S, Bagsvaerd (DK)

(21) Appl. No.: 13/812,194

(22) PCT Filed: Aug. 2, 2011
(86) PCT No.: PCT/US11/46212

§ 371 Date: Mar. 4, 2013

Related U.S. Application Data
(60) Provisional application No. 61/369,870, filed on Aug. 2, 2010.

(51) Int. Cl. C12P 7/14

(52) U.S. Cl. CPC C12P 7/14
USPC 435/43; 435/162; 435/158; 435/160; 435/157; 435/145; 435/140; 435/139; 435/137; 435/138; 435/148; 435/150; 435/110; 435/168; 435/127; 435/67; 435/254.2; 435/254.21; 435/254.23; 435/254.22; 435/252.33; 435/252.3; 435/252.32; 435/252.31

(57) ABSTRACT

The invention relates to a process of fermenting plant material in a fermentation medium into a fermentation product using a fermenting organism, wherein one or more deamidases are present in the fermentation medium.

### FIELD OF THE INVENTION

The present invention relates to methods of producing a fermentation product from plant material using one or more fermenting organisms; compositions; transgenic plants; and modified fermenting organisms, that can be used in methods and/or processes of the invention.

### BACKGROUND ART

A vast number of commercial products that are difficult to produce synthetically are today produced by fermenting organisms. Such products include alcohols (e.g., ethanol, methanol, butanol, 1,3-propanediol); organic acids (e.g., citric acid, acetic acid, itaconic acid, lactic acid, gluconic acid, gluconate, lactic acid, succinic acid, 2,5-diketo-D-gluconic acid); ketones (e.g., acetone); amino acids (e.g., glutamic acid); gases (e.g., H₂ and CO₂), and more complex compounds, including, for example, antibiotics (e.g., penicillin and tetracycline); enzymes; vitamins (e.g., riboflavin, B₁₂, beta-carotene); and hormones. Fermentation is also commonly used in the consumable alcohol (e.g., beer and wine), dairy (e.g., in the production of yogurt and cheese), leather, and tobacco industries.

A vast number of processes of producing fermentation products, such as ethanol, by fermentation of sugars provided by degradation of starch-containing material are known in the art.

However, production of fermentation products, such as ethanol, from such plant materials is still too costly. Therefore, there is a need for providing processes that can increase the yield of the fermentation product and thereby reducing the production costs.

Yong et al., 2004, J. Agric. Food Chem. 52: 7094-7100 disclose that zein can be solubilized by the action of a deamidase (protein-glutaminase). Similar observations on wheat gluten were disclosed in Yong et al., 2006, J. Agric. Food Chem. 54: 6034-6040.

U.S. Pat. No. 7,279,298 discloses the use of a Chryseobacterium sp. deamidase for solubilizing plant/animal proteins with a molecular weight ≧5000 Da.

It is an object of the present invention to provide an improved process for producing a fermentation product.

### SUMMARY OF THE INVENTION

The present invention relates to a process of producing a fermentation product from gelatinized or ungelatinized starch-containing material. In particular, the present invention relates to a process of producing a fermentation product, comprising:

(a) converting a starch-containing material to dextrins with an alpha-amylase;

(b) saccharifying the dextrins to a sugar with a glucoamylase;

(c) adding a deamidase; and

(d) fermenting the sugar using a fermenting organism.

The present invention also relates to a composition comprising one or more deamidases and one or more enzymes selected from the group consisting of alpha-amylases, beta-amylases, glucoamylases, maltogenic amylases, and pullulanases.

The present invention also relates to the use of deamidase or compositions of the invention in a fermentation process to produce a fermentation product.

The present invention also relates to modified fermenting organisms, which have been transformed with a polynucleotide encoding a deamidase, wherein the fermenting organism is capable of expressing the deamidase at fermentation conditions.

### DETAILED DESCRIPTION OF THE INVENTION

The present invention relates to a process of producing a fermentation product, comprising:

(a) converting a starch-containing material to dextrins with an alpha-amylase;

(b) saccharifying the dextrins to a sugar with a glucoamylase;

(c) adding a deamidase; and

(d) fermenting the sugar using a fermenting organism.

### Fermentation Products

The term “fermentation product” means a product produced by a method or process including fermenting using a fermenting organism. Fermentation products contemplated according to the invention include alcohols (e.g., ethanol, methanol, butanol); organic acids (e.g., citric acid, acetic acid, itaconic acid, lactic acid, succinic acid, gluconic acid); ketones (e.g., acetone); amino acids (e.g., glutamic acid); gases (e.g., H₂ and CO₂); antibiotics (e.g., penicillin and tetracycline); enzymes; vitamins (e.g., riboflavin, B₁₂, beta-carotene); and hormones. In a preferred embodiment the fermentation product is ethanol, e.g., fuel ethanol; drinking ethanol, i.e., potable neutral spirits; or industrial ethanol or products used in the consumable alcohol industry (e.g., beer and wine), dairy industry (e.g., fermented dairy products), leather industry and tobacco industry. Preferred beer types comprise ales, stouts, porters, lagers, bitters, malt liquors, happoushu, high-alcohol beer, low-alcohol beer, low-calorie beer or light beer.

Preferred fermentation processes include alcohol fermentation processes. The fermentation product, such as ethanol, obtained according to the invention, may preferably be used as fuel. However, in the case of ethanol it may also be used as potable ethanol.

### Starch-Containing Materials

Any suitable starch-containing starting material, including granular starch (raw uncooked starch), may be used according to the present invention. The starting material is generally selected based on the desired fermentation product. Examples of starch-containing starting materials, suitable for use in methods or processes of the present invention, include barley, beans, cassaya, cereals, corn, milo, peas, potatoes, rice, rye, sago, sorghum, sweet potatoes, tapioca, wheat, and whole grains, or any mixture thereof. The starch-containing material may also be a waxy or non-waxy type of corn and barley.

The term “granular starch” means raw uncooked starch, i.e., starch in its natural form found in cereal, tubers or grains. Starch is formed within plant cells as tiny granules insoluble in water. When put in cold water, the starch granules may absorb a small amount of the liquid and swell. At temperatures up to 50° C. to 75° C. the swelling may be reversible. However, with higher temperatures an irreversible swelling called “gelatinization” begins. Granular starch to be processed may be a highly refined starch quality, preferably at least 90%, at least 95%, at least 97% or at least 99.5% pure or it may be a more crude starch-containing materials comprising (e.g., milled) whole grains including non-starch fractions such as germ residues and fibers. The raw material, such as whole grains, may be reduced in particle size, e.g., by milling, in order to open up the structure and allowing for further processing. Two processes are preferred according to the invention: wet and dry milling. In dry milling whole kernels are milled and used. Wet milling gives a good separation of germ and meal (starch granules and protein) and is often applied at locations where the starch hydrolyzate is used in production of, e.g., syrups. Both dry and wet milling is well known in the art of starch processing and is equally contemplated for a process of the invention. In an embodiment the particle size is reduced to between 0.05-3.0 mm, preferably 0.1-0.5 mm, or so that at least 30%, preferably at least 50%, more preferably at least 70%, even more preferably at least 90% of the starch-containing material fit through a sieve with a 0.05-3.0 mm screen, preferably 0.1-0.5 mm screen.

Processes for Producing Fermentation Products from Gelatinized Starch-Containing Material

In this aspect of the invention, the starch-containing material is converted to dextrins with an alpha-amylase in a liquefaction step, which is then followed by saccharification and fermentation. The saccharification and fermentation steps can be performed sequentially or simultaneously.

The saccharification and fermentation steps may be carried out either sequentially or simultaneously. The deamidase may be added during liquefaction, saccharification, fermentation, or simultaneous saccharification/fermentation.

The liquefaction is preferably carried out in the presence of an alpha-amylase, preferably a bacterial alpha-amylase and/or acid fungal alpha-amylase. In an embodiment, a pullulanase is added during liquefaction. The fermenting organism is preferably yeast, preferably a strain of Saccharomyces cerevisiae.

Liquefaction may be carried out as a three-step hot slurry process. The slurry is heated to between 60-95° C., e.g., 80-85° C., and an alpha-amylase is added to initiate liquefaction (thinning). Then the slurry may be jet-cooked at a temperature between 95-140° C., e.g., 105-125° C., for about 1-15 minutes, e.g., about 3-10 minutes, especially around 5 minutes. The slurry is cooled to 60-95° C. and more alpha-amylase is added to finalize the hydrolysis (secondary liquefaction). The liquefaction process is usually carried out at a pH of 4.5-6.5, in particular at a pH from 5 to 6. All of the alpha-amylases may be added as a single dose, e.g., before jet cooking.

The saccharification step may be carried out using conditions well known in the art. For instance, a full saccharification step may last up to from about 24 to about 72 hours, however, it is also common only to do a pre-saccharification of typically 40-90 minutes at a temperature between 30-65° C., typically about 60° C., followed by complete saccharification during fermentation in a simultaneous saccharification and fermentation (SSF) process. Saccharification is typically carried out at a temperature in the range of 20-75° C., e.g., 40-70° C., typically around 60° C., and at a pH between about 4 and 5, normally at about pH 4.5.

In an embodiment, saccharification and fermentation are performed simultaneously (SSF), in which there is no holding stage for the saccharification, meaning that fermenting organism(s), such as yeast, and enzyme(s), including the deamidase, may be added together. SSF is typically carried out at a temperature from 20° C. to 40° C., e.g., 26° C. to 34° C., preferably around 32° C., when the fermentation organism is yeast, such as a strain of Saccharomyces cerevisiae, and the desired fermentation product is ethanol.

Other fermentation products may be fermented at conditions and temperatures, well known to persons skilled in the art, suitable for the fermenting organism in question. According to the invention the temperature may be adjusted up or down during fermentation.

In a particular embodiment, the process of the invention further comprises, prior to the conversion of a starch-containing material to dextrins, the steps of:

x) reducing the particle size of the starch-containing material;

y) forming a slurry comprising the starch-containing material and water.

Methods for reducing the particle size of the starch containing material are known to those skilled in the art. In an embodiment, the starch-containing material is milled to reduce the particle size.

The aqueous slurry may contain from 10-55 wt. % dry solids (DS), preferably 25-45 wt. % dry solids (DS), more preferably 30-40 wt. % dry solids (DS) of starch-containing material. The slurry is heated to above the gelatinization temperature and an alpha-amylase, preferably a bacterial and/or acid fungal alpha-amylase, may be added to initiate liquefaction or thinning. The slurry may be jet-cooked to further gelatinize the slurry before being subjected to an alpha-amylase in step i) of the invention.

Processes for Producing Fermentation Products from Un-Qelatinized Starch-Containing Material

In this aspect, the invention relates to processes for producing a fermentation product from starch-containing material without gelatinization (often referred to as “without cooking”) of the starch-containing material. In one embodiment a process of the invention includes saccharifying (e.g., milled) starch-containing material, e.g., granular starch, below the initial gelatinization temperature, preferably in the presence of an alpha-amylase and/or a carbohydrate-source generating enzyme(s) to produce sugars that can be fermented into the desired fermentation product by a suitable fermenting organism.

In an embodiment of the invention, the desired fermentation product, preferably ethanol, is produced from un-gelatinized (i.e., uncooked), preferably milled, cereal grains, such as corn.

Accordingly, in this aspect the invention relates to processes of producing a fermentation product from starch-containing material comprising the steps of:

(a) saccharifying starch-containing material at a temperature below the initial gelatinization temperature of said starch-containing material;

(b) fermenting using one or more fermenting organisms, wherein fermentation is carried out in the presence of one or more deamidases.

In a preferred embodiment steps (a) and (b) are carried out simultaneously (i.e., one-step fermentation) or sequentially.

The fermentation product, such as especially ethanol, may be recovered after fermentation, e.g., by distillation. Typically amylase(s), such as glucoamylase(s) and/or other carbohydrate-source generating enzymes and/or alpha-amylase(s), are present during fermentation.

The term “initial gelatinization temperature” means the lowest temperature at which starch gelatinization commences. In general, starch heated in water begins to gelatinize between about 50° C. and 75° C.; the exact temperature of gelatinization depends on the specific starch and can readily be determined by the skilled artisan. Thus, the initial gelatinization temperature may vary according to the plant species, to the particular variety of the plant species as well as with the growth conditions. In the context of this invention the initial gelatinization temperature of a given starch-containing material may be determined as the temperature at which birefringence is lost in 5% of the starch granules using the method described by Gorinstein and Lii, 1992, Starch/Stärke 44(12): 461-466.

Before step (a) a slurry of starch-containing material, such as granular starch, having 10-55 wt. % dry solids (DS), preferably 25-45 wt. % dry solids, more preferably 30-40 wt. dry solids of starch-containing material may be prepared. The slurry may include water and/or process water, such as stillage (backset), scrubber water, evaporator condensate or distillate, side-stripper water from distillation, or process water from other fermentation product plants. Because the process of the invention is carried out below the initial gelatinization temperature and thus no significant viscosity increase takes place, high levels of stillage may be used if desired. In an embodiment the aqueous slurry contains from about 1 to about 70 vol. %, preferably 15-60 vol. %, especially from about 30 to 50 vol. % water and/or process waters, such as stillage (backset), scrubber water, evaporator condensate or distillate, side-stripper water from distillation, or process water from other fermentation product plants, or combinations thereof, or the like.

The starch-containing material may be prepared by reducing the particle size, preferably by dry or wet milling, to 0.05-3.0 mm, preferably 0.1-0.5 mm. After being subjected to a method or process of the invention at least 85%, at least 86%, at least 87%, at least 88%, at least 89%, at least 90%, at least 91%, at least 92%, at least 93%, at least 94%, at least 95%, at least 96%, at least 97%, at least 98%, or preferably at least 99% of the dry solids in the starch-containing material is converted into a soluble starch hydrolyzate.

The process of this aspect of the invention is conducted at a temperature below the initial gelatinization temperature. When step (a) is carried out separately from fermentation step (b) the temperature typically lies in the range between 30-75° C., preferably in the range from 45-60° C. The following separate fermentation step (b) is then carried out at a temperature suitable for the fermenting organism, which typically is in the range between 25-40° C. when the fermenting organism is yeast.

In a preferred embodiment step (a) and step (b) are carried out as a simultaneous saccharification and fermentation process. In such embodiment the process is typically carried out at a temperature between 25° C. and 40° C., such as between 29° C. and 35° C., such as between 30° C. and 34° C., such as around 32° C., when the fermenting organism is yeast. One skilled in the art can easily determine which process conditions are suitable.

In an embodiment fermentation is carried out so that the sugar level, such as glucose level, is kept at a low level, such as below 6 wt. %, such as below about 3 wt. %, such as below about 2 wt. %, such as below about 1 wt. %, such as below about 0.5 wt. %, or below 0.25% wt. %, such as below about 0.1 wt. %. Such low levels of sugar can be accomplished by simply employing adjusted quantities of enzyme and fermenting organism. A skilled person in the art can easily determine which doses/quantities of enzyme and fermenting organism to use. The employed quantities of enzyme and fermenting organism may also be selected to maintain low concentrations of maltose in the fermentation broth. For instance, the maltose level may be kept below about 0.5 wt. %, such as below about 0.2 wt. %.

The process of the invention may be carried out at a pH from about 3 and 7, preferably from pH 3.5 to 6, or more preferably from pH 4 to 5.

### Saccharification and Fermentation

In one embodiment of the present invention, saccharification and fermentation are carried out as a simultaneous saccharification and fermentation step (SSF). In general this means that combined/simultaneous saccharification and fermentation are carried out at conditions (e.g., temperature and/or pH) suitable, preferably optimal, for the fermenting organism(s) in question.

In another embodiment saccharification and fermentation are carried out as a hybrid saccharification and fermentation, which typically begins with a separate partial hydrolysis step and ends with a simultaneous hydrolysis and fermentation step. The separate partial hydrolysis step is an enzymatic saccharification step typically carried out at conditions (e.g., at higher temperatures) suitable, preferably optimal, for the hydrolyzing enzyme(s) in question. The subsequent simultaneous saccharification and fermentation step is typically carried out at conditions suitable for the fermenting organism(s) (often at lower temperatures than the separate hydrolysis step).

In another embodiment, the saccharification and fermentation steps may also be carried out as separate saccharification and fermentation, where the saccharification is taken to completion before initiation of fermentation.

### Fermenting Organisms

The term “fermenting organism” refers to any organism, including bacterial and fungal organisms, including yeast and filamentous fungi, suitable for producing a desired fermentation product. Suitable fermenting organisms according to the invention are able to ferment, i.e., convert fermentable sugars, such as glucose, fructose, maltose, xylose, mannose or arabinose, directly or indirectly into the desired fermentation product.

Examples of fermenting organisms include fungal organisms such as yeast. Preferred yeast includes strains of the genus Saccharomyces, in particular strains of Saccharomyces cerevisiae or Saccharomyces uvarum; a strain of Pichia, preferably Pichia stipitis such as Pichia stipitis CBS 5773 or Pichia pastoris; a strain of the genus Candida, in particular a strain of Candida utilis, Candida arabinofermentans, Candida diddensii, Candida sonorensis, Candida shehatae, Candida tropicalis, or Candida boidinii. Other fermenting organisms include strains of Hansenula, in particular Hansenula polymorpha or Hansenula anomala; Kluyveromyces, in particular Kluyveromyces fragilis or Kluyveromyces marxianus; and Schizosaccharomyces, in particular Schizosaccharomyces pombe.

Preferred bacterial fermenting organisms include strains of Escherichia, in particular Escherichia coli, strains of Zymomonas, in particular Zymomonas mobilis, strains of Zymobacter, in particular Zymobactor palmae, strains of Klebsiella in particular Klebsiella oxytoca, strains of Leuconostoc, in particular Leuconostoc mesenteroides, strains of Clostridium, in particular Clostridium butyricum, strains of Enterobacter, in particular Enterobacter aerogenes and strains of Thermoanaerobacter, in particular Thermoanaerobacter BG1 L1 (Appl. Microbiol. Biotech. 77: 61-86) and Thermoanarobacter ethanolicus, Thermoanaerobacter thermosaccharolyticum, or Thermoanaerobacter mathranii. Strains of Lactobacillus are also envisioned as are strains of Corynebacterium glutamicum R, Bacillus thermoglucosidaisus, and Geobacillus thermoglucosidasius.

In an embodiment the fermenting organism is a C6 sugar fermenting organism, such as a strain of, e.g., Saccharomyces cerevisiae.

In one embodiment the fermenting organism is added to the fermentation medium so that the viable fermenting organism, such as yeast, count per mL of fermentation medium is in the range from 10⁵ to 10¹², preferably from 10⁷ to 10¹⁰, especially about 5×10⁷.

Yeast is the preferred fermenting organism for ethanol fermentation. Preferred are strains of Saccharomyces, especially strains of the species Saccharomyces cerevisiae, preferably strains which are resistant towards high levels of ethanol, i.e., up to, e.g., about 10, about 12, about 15 or about 20 vol. % or more ethanol.

Commercially available yeast includes, e.g., RED START™ and ETHANOL RED™ yeast (available from Fermentis/Lesaffre, USA), FALI (available from Fleischmann's Yeast, USA), SUPERSTART and THERMOSACC™ fresh yeast (available from Ethanol Technology, Wis., USA), BIOFERM AFT and XR (available from NABC—North American Bioproducts Corporation, Ga., USA), GERT STRAND (available from Gert Strand AB, Sweden), and FERMIOL (available from DSM Specialties).

According to the invention the fermenting organism capable of producing a desired fermentation product from fermentable sugars, including glucose, fructose, maltose, xylose, mannose, or arabinose, is preferably grown under precise conditions at a particular growth rate. When the fermenting organism is introduced into/added to the fermentation medium the inoculated fermenting organism pass through a number of stages. Initially growth does not occur. This period is referred to as the “lag phase” and may be considered a period of adaptation. During the next phase referred to as the “exponential phase” the growth rate gradually increases. After a period of maximum growth the rate ceases and the fermenting organism enters “stationary phase”. After a further period of time the fermenting organism enters the “death phase” where the number of viable cells declines.

In one embodiment the deamidase is added to the fermentation medium when the fermenting organism is in lag phase.

In another embodiment the deamidase is added to the fermentation medium when the fermenting organism is in exponential phase.

In another embodiment deamidase is added to the fermentation medium when the fermenting organism is in stationary phase.

### Fermentation

The plant starting material used in fermenting methods or processes of the invention is a starch-containing material. The fermentation conditions are determined based on, e.g., the kind of plant material, the available fermentable sugars, the fermenting organism(s) and/or the desired fermentation product. One skilled in the art can easily determine suitable fermentation conditions. The fermentation may according to the invention be carried out at conventionally used conditions. Preferred fermentation processes are anaerobic processes.

The methods or processes of the invention may be performed as a batch or as a continuous process. Fermentations of the invention may be conducted in an ultrafiltration system wherein the retentate is held under recirculation in the presence of solids, water, and the fermenting organism, and wherein the permeate is the desired fermentation product containing liquid. Equally contemplated are methods/processes conducted in continuous membrane reactors with ultrafiltration membranes and where the retentate is held under recirculation in presence of solids, water, and the fermenting organism(s) and where the permeate is the fermentation product containing liquid.

After fermentation the fermenting organism may be separated from the fermented slurry and recycled.

### Fermentation Medium

The phrase “fermentation media” or “fermentation medium” refers to the environment in which fermentation is carried out and comprises the fermentation substrate, that is, the carbohydrate source that is metabolized by the fermenting organism(s), and may include the fermenting organism(s).

The fermentation medium may comprise nutrients and growth stimulator(s) for the fermenting organism(s). Nutrient and growth stimulators are widely used in the art of fermentation and include nitrogen sources, such as ammonia; vitamins and minerals, or combinations thereof.

Following fermentation, the fermentation media or fermentation medium may further comprise the fermentation product.

### Fermentation of Starch-Derived Sugars

Different kinds of fermenting organisms may be used for fermenting sugars derived from starch-containing material. Fermentations are conventionally carried out using yeast, e.g., of the genus Saccharomyces, such as a strain of Saccharomyces cerevisae, as the fermenting organism. However, bacteria and filamentous fungi may also be used as fermenting organisms. Some bacteria have higher fermentation temperature optimum than, e.g., Saccharomyces cerevisae. Therefore, fermentations may in such cases be carried out at temperatures as high as 75° C., e.g., between 40-70° C., such as between 50-60° C. However, bacteria with a significantly lower temperature optimum down to around room temperature (around 20° C.) are also known. Examples of suitable fermenting organisms can be found in the “Fermenting Organisms” section above.

For ethanol production using yeast, the fermentation may in one embodiment go on for 24 to 96 hours, in particular for 35 to 60 hours. In an embodiment the fermentation is carried out at a temperature between 20 to 40° C., preferably 26 to 34° C., in particular around 32° C. In an embodiment the pH is from pH 3 to 6, preferably around pH 4 to 5.

Other fermentation products may be fermented at temperatures known to the skilled person in the art to be suitable for the fermenting organism in question.

Fermentation is typically carried out at a pH in the range between 3 and 7, preferably from pH 3.5 to 6, such as around pH 5. Fermentations are typically ongoing for 24-96 hours.

### Recovery

Subsequent to fermentation, the fermentation product may be separated from the fermentation medium. The fermentation medium may be distilled to extract the desired fermentation product or the desired fermentation product may be extracted from the fermentation medium by micro or membrane filtration techniques. Alternatively, the fermentation product may be recovered by stripping. Methods for recovery are well known in the art.

### Enzymes

Even if not specifically mentioned in context of a method or process of the invention, it is to be understood that enzyme(s) is(are) used in an “effective amount”.

### Deamidases

According to the present invention, a deamidase is an enzyme which acts upon side chain amido groups in free amino acids (asparagine and glutamine) or in the amino acid sequence of a peptide and/or polypeptide, and thereby releases side chain carboxyl groups and ammonia. Examples of deamidases include asparaginases (EC 3.5.1.1), glutaminases (EC 3.5.1.2), peptidyl-glutaminases (EC 3.5.1.43), and protein-glutamine glutaminases (EC 3.5.1.44). Deamidases may be derived from Cytophagales or Actinomycetes, more particularly from Aureobacterium, Chryseobacterium, Empedobacter, Flavobacterium, Myroides, or Sphingobacterium. Deamidases may also be derived from Bacillus, e.g., Bacillus amyloliquefaciens.

In an embodiment, the deamidase has at least 70% sequence identity to SEQ ID NO: 6 disclosed in U.S. Pat. No. 6,251,651, e.g., at least 75%, at least 80%, at least 85%, at least 90%, at least 91%, at least 92%, at least 93%, at least 94%, at least 95%, at least 96%, at least 97%, at least 98%, at least 99%, and at least 100% sequence identity.

The deamidase may be added/introduced during the conversion of the starch material to dextrins, the saccharification of the dextrins, or fermentation. The deamidase may be from an exogenous source, and/or may be produced, e.g., in situ by overexpression of deamidase by the fermenting organism(s). The latter can be accomplished by preparing modified fermenting organisms, such as yeast, that are capable of expressing a deamidase, e.g., by transforming the yeast with one or more deamidase encoding genes or by introducing a promoter that increases the expression of an endogenous deamidase gene. Techniques for introducing deamidase genes into a fermenting organism, such as yeast, and/or over-expressing a deamidase gene in a fermenting organism are known in the art. A deamidase may also be present/introduced into the fermentation medium in the form of a transgenic plant material containing and/or expressing deamidase.

### Alpha-Amylases

According to the invention any alpha-amylase may be used, such as of fungal, bacterial or plant origin. In a preferred embodiment the alpha-amylase is an acid alpha-amylase, e.g., acid fungal alpha-amylase or acid bacterial alpha-amylase. The term “acid alpha-amylase” means an alpha-amylase (E.C. 3.2.1.1) which added in an effective amount has activity optimum at a pH in the range of 3 to 7, preferably from 3.5 to 6, or more preferably from 4-5.

### Bacterial Alpha-Amylases

According to the invention a bacterial alpha-amylase is preferably derived from the genus Bacillus.

In a preferred embodiment the Bacillus alpha-amylase is derived from a strain of Bacillus amyloliquefaciens, Bacillus licheniformis, Bacillus stearothermophilus, or Bacillus subtilis, but may also be derived from other Bacillus sp. Specific examples of contemplated alpha-amylases include the Bacillus amyloliquefaciens alpha-amylase SEQ ID NO: 5 in WO 99/19467, the Bacillus lichenifonnis alpha-amylase shown in SEQ ID NO: 4 in WO 99/19467, and the Bacillus stearothermophilus alpha-amylase shown in SEQ ID NO: 3 in WO 99/19467 (all sequences are hereby incorporated by reference). In an embodiment the alpha-amylase may be an enzyme having a degree of identity of at least 60%, e.g., at least 70%, at least 80%, at least 90%, at least 95%, at least 96%, at least 97%, at least 98% or at least 99% to any one of the sequences shown in SEQ ID NOS: 3, 4 or 5, respectively, in WO 99/19467.

The Bacillus alpha-amylase may also be a variant and/or hybrid, especially one described in any of WO 96/23873, WO 96/23874, WO 97/41213, WO 99/19467, WO 00/60059, and WO 02/10355 (all documents are hereby incorporated by reference). Specific alpha-amylase variants are disclosed in U.S. Pat. Nos. 6,093,562, 6,187,576, and 6,297,038 (hereby incorporated by reference) and include Bacillus stearothermophilus alpha-amylase (BSG alpha-amylase) variants having a deletion of one or two amino acids at positions R179 to G182, preferably a double deletion disclosed in WO 96/23873—see, e.g., page 20, lines 1-10 (hereby incorporated by reference), preferably corresponding to delta(181-182) compared to the amino acid sequence of Bacillus stearothermophilus alpha-amylase set forth in SEQ ID NO:3 disclosed in WO 99/19467 or the deletion of amino acids R179 and G180 using SEQ ID NO:3 in WO 99/19467 for numbering (which reference is hereby incorporated by reference). Even more preferred are Bacillus alpha-amylases, especially Bacillus stearothermophilus alpha-amylases, which have a double deletion corresponding to delta(181-182) and further comprise a N193F substitution (also denoted 1181*+G182*+N193F) compared to the wild-type BSG alpha-amylase amino acid sequence set forth in SEQ ID NO:3 disclosed in WO 99/19467.

### Bacterial Hybrid Alpha-Amylases

A hybrid alpha-amylase specifically contemplated comprises 445 C-terminal amino acid residues of the Bacillus lichenifonnis alpha-amylase (shown in SEQ ID NO: 4 of WO 99/19467) and the 37 N-terminal amino acid residues of the alpha-amylase derived from Bacillus amyloliquefaciens (shown in SEQ ID NO: 5 of WO 99/19467), with one or more, especially all, of the following substitutions: G48A+T49I+G107A+H156Y+A181T+N190F+1201F+A209V+Q264S (using the Bacillus licheniformis numbering in SEQ ID NO: 4 of WO 99/19467). Also preferred are variants having one or more of the following mutations (or corresponding mutations in other Bacillus alpha-amylase backbones): H154Y, A181T, N190F, A209V and Q264S and/or the deletion of two residues between positions 176 and 179, preferably the deletion of E178 and G179 (using SEQ ID NO: 5 WO 99/19467 for position numbering).

In an embodiment the bacterial alpha-amylase is dosed in an amount of 0.0005-5 KNU per g DS (dry solids), preferably 0.001-1 KNU per g DS, such as around 0.050 KNU per g DS.

### Fungal Alpha-Amylases

Fungal alpha-amylases include alpha-amylases derived from a strain of the genus Aspergillus, such as, Aspergillus kawachii, Aspergillus niger and Aspergillus oryzae alpha-amylases.

A preferred acidic fungal alpha-amylase is an alpha-amylase which exhibits a high identity, i.e., at least 70%, at least 75%, at least 80%, at least 85%, at least 90%, at least 95%, at least 96%, at least 97%, at least 98%, at least 99% or even 100% identity to the mature part of the amino acid sequence shown in SEQ ID NO: 10 in WO 96/23874.

Another preferred acid alpha-amylase is derived from a strain Aspergillus niger. In a preferred embodiment the acid fungal alpha-amylase is the one from Aspergillus niger disclosed as “AMYA_ASPNG” in the Swiss-prot/TeEMBL database under the primary accession no. P56271 and described in WO 89/01969 (Example 3—incorporated by reference). An acid fungal alpha-amylase derived from Aspergillus niger is SP288 (available from Novozymes A/S, Denmark).

Other contemplated wild-type alpha-amylases include those derived from a strain of Meripilus and Rhizomucor, preferably a strain of Meripilus giganteus or Rhizomucor pusillus (WO 2004/055178 incorporated by reference).

In a preferred embodiment the alpha-amylase is derived from Aspergillus kawachii and disclosed by Kaneko et al., 1996, J. Ferment. Bioeng. 81: 292-298, “Molecular-cloning and determination of the nucleotide-sequence of a gene encoding an acid-stable alpha-amylase from Aspergillus kawachii”; and further as EMBL: #AB008370.

The fungal alpha-amylase may also be a wild-type enzyme comprising a starch-binding domain (SBD) and an alpha-amylase catalytic domain (i.e., non-hybrid), or a variant thereof. In an embodiment the wild-type alpha-amylase is derived from a strain of Aspergillus kawachii.

### Fungal Hybrid Alpha-Amylases

In a preferred embodiment the fungal acid alpha-amylase is a hybrid alpha-amylase. Examples of fungal hybrid alpha-amylases include the ones disclosed in WO 2005/003311, U.S. Patent Application Publication No. 2005/0054071 (Novozymes), and WO 2006/069290 (Novozymes), which are hereby incorporated by reference. A hybrid alpha-amylase may comprise an alpha-amylase catalytic domain (CD) and a carbohydrate-binding domain/module (CBM), such as a starch binding domain, and optionally a linker.

Specific examples of hybrid alpha-amylases include those disclosed in Tables 1 to 5 of the examples in WO 2006/069290 including Fungamyl variant with the catalytic domain JA118 and Athelia rolfsii SBD (SEQ ID NO:100 in U.S. provisional application No. 60/638,614), Rhizomucor pusillus alpha-amylase with Athelia rolfsii AMG linker and SBD (SEQ ID NO:101 in U.S. provisional application No. 60/638,614), Rhizomucor pusillus alpha-amylase with Aspergillus niger glucoamylase linker and SBD (which is disclosed in Table 5 as a combination of amino acid sequences SEQ ID NO:20, SEQ ID NO:72 and SEQ ID NO:96 in U.S. application Ser. No. 11/316,535) or as V039 in Table 5 in WO 2006/069290, and Meripilus giganteus alpha-amylase with Athelia rolfsii glucoamylase linker and SBD (SEQ ID NO:102 in WO 2006/069290). Other hybrid alpha-amylases are listed in Tables 3, 4, 5, and 6 in Example 4 in U.S. application Ser. No. 11/316,535 and WO 2006/069290 (which are hereby incorporated by reference).

Other specific examples of hybrid alpha-amylases include those disclosed in U.S. Patent Application Publication No. 2005/0054071, including hybrid Aspergillus niger alpha-amylases with a fungal starch-binding domain with or without a linker, preferably those disclosed in Table 3 on page 15, such as Aspergillus niger alpha-amylase with Aspergillus kawachii alpha-amylase linker and Aspergillus kawachii alpha-amylase starch binding domain (JA001) or Aspergillus niger alpha-amylase with Aspergillus kawachii alpha-amylase linker and Althea rolfsii glucoamylase starch binding domain (JA004).

Other alpha-amylases exhibit a high degree of sequence identity to any of above mentioned alpha-amylases, i.e., at least 70%, at least 75%, at least 80%, at least 85%, at least 90%, at least 95%, at least 96%, at least 97%, at least 98%, at least 99% or even 100% identity to the mature enzyme sequences disclosed above.

An acid alpha-amylase may according to the invention be added in an amount of 0.001 to 10 AFAU/g DS, preferably from 0.01 to 5 AFAU/g DS, especially 0.3 to 2 AFAU/g DS or 0.001 to 1 FAU-F/g DS, preferably 0.01 to 1 FAU-F/g DS.

### Commercial Alpha-Amylase Products

Preferred commercial compositions comprising alpha-amylase include MYCOLASE™ from DSM (Gist Brocades), BAN™, TERMAMYL™ SC, FUNGAMYLT™, LIQUOZYME™ X, LIQUOZYME™ SC and SAN™ SUPER, SAN™ EXTRA L (Novozymes A/S) and CLARASE™ L-40,000, DEX-LO™, SPEZYME™ FRED, SPEZYME™ AA, SPEZYME™ DELTA AA, GC358, GC980, and SPEZYME™ RSL (Danisco A/S), and the acid fungal alpha-amylase sold under the trade name SP288 (available from Novozymes A/S, Denmark).

### Carbohydrate-Source Generating Enzymes

The term “carbohydrate-source generating enzyme” includes glucoamylase (a glucose generator), beta-amylase and maltogenic amylase (both maltose generators) and also pullulanase and alpha-glucosidase. A carbohydrate-source generating enzyme is capable of producing a carbohydrate that can be used as an energy-source by the fermenting organism(s) in question, for instance, when used in a process of the invention for producing a fermentation product, such as ethanol. The generated carbohydrate may be converted directly or indirectly to the desired fermentation product, preferably ethanol. According to the invention a mixture of carbohydrate-source generating enzymes may be used. Especially contemplated blends are mixtures comprising at least a glucoamylase and an alpha-amylase, especially an acid amylase, even more preferred an acid fungal alpha-amylase.

The ratio between glucoamylase activity (AGU) and acid fungal alpha-amylase activity (FAU-F) (i.e., AGU per FAU-F) may in a preferred embodiment of the invention be between 0.1 and 100 AGU/FAU-F, in particular between 2 and 50 AGU/FAU-F, such as in the range from 10-40 AGU/FAU-F, especially when doing one-step fermentation (Raw Starch Hydrolysis —RSH), i.e., when saccharification in step (a) and fermentation in step (b) are carried out simultaneously (i.e., without a liquefaction step).

In a conventional starch-to-ethanol process (i.e., including a liquefaction step (a)) the ratio may preferably be as defined in EP 140,410-B1, especially when saccharification and fermentation are carried out simultaneously.

### Glucoamylases

A glucoamylase may be derived from any suitable source, e.g., derived from a microorganism or a plant. Preferred glucoamylases are of fungal or bacterial origin, selected from the group consisting of Aspergillus glucoamylases, in particular Aspergillus niger G1 or G2 glucoamylase (Boel et al., 1984, EMBO J. 3(5): 1097-1102), or variants thereof, such as those disclosed in WO 92/00381, WO 00/04136 and WO 01/04273 (from Novozymes, Denmark); the A. awamori glucoamylase disclosed in WO 84/02921, Aspergillus oryzae glucoamylase (Hata et al., 1991, Agric. Biol. Chem. 55(4): 941-949), or variants or fragments thereof. Other Aspergillus glucoamylase variants include variants with enhanced thermal stability: G137A and G139A (Chen et al., 1996, Prot. Eng. 9: 499-505); D257E and D293E/Q (Chen et al., 1995, Prot. Eng. 8: 575-582); N182 (Chen et al., 1994, Biochem. J. 301: 275-281); disulphide bonds, A246C (Fierobe et al., 1996, Biochemistry 35: 8698-8704; and introduction of Pro residues in position A435 and S436 (Li et al., 1997, Protein Eng. 10: 1199-1204.

Other glucoamylases include Athelia rolfsii (previously denoted Corticium rolfsii) glucoamylase (see U.S. Pat. No. 4,727,026 and (Nagasaka et al., 1998, Appl. Microbiol. Biotechnol. 50: 323-330), Talaromyces glucoamylases, in particular derived from Talaromyces duponti, Talaromyces emersonii (WO 99/28448), Talaromyces leycettanus (U.S. Pat. No. Re. 32,153), and Talaromyces thermophilus (U.S. Pat. No. 4,587,215).

Bacterial glucoamylases include glucoamylases from Clostridium, in particular C. thermoamylolyticum (EP 135138), and C. thermohydrosulfuricum (WO 86/01831), Trametes cingulata, Pachykytospora papyracea, and Leucopaxillus giganteus, all disclosed in WO 2006/069289; or Peniophora rufomarginata disclosed in PCT/US2007/066618; or a mixture thereof. A hybrid glucoamylase may be used in the present invention. Examples of hybrid glucoamylases are disclosed in WO 2005/045018. Specific examples include the hybrid glucoamylase disclosed in Tables 1 and 4 of Example 1 (which hybrids are hereby incorporated by reference).

The glucoamylase may have a high degree of sequence identity to any of above mentioned glucoamylases, i.e., at least 70%, at least 75%, at least 80%, at least 85%, at least 90%, at least 95%, at least 96%, at least 97%, at least 98%, at least 99% or even 100% identity to the mature enzymes sequences mentioned above.

Commercially available compositions comprising glucoamylase include AMG 200L; AMG 300 L; SAN™ SUPER, SAN™ EXTRA L, SPIRIZYME™ PLUS, SPIRIZYME™ FUEL, SPIRIZYME™ B4U, SPIRIZYME ULTRA™ and AMG™ E (from Novozymes A/S, Denmark); OPTIDEX™ 300, GC480™ and GC147™ (from Danisco US Inc., USA); AMIGASE™ and AMIGASE™ PLUS (from DSM); G-ZYME™ G900, G-ZYME™ and G990 ZR (from Danisco US Inc.).

Glucoamylases may be added in an amount of 0.02-20 AGU/g DS, preferably 0.1-10 AGU/g DS, especially between 1-5 AGU/g DS, such as 0.1-2 AGU/g DS, such as 0.5 AGU/g DS or in an amount of 0.0001-20 AGU/g DS, preferably 0.001-10 AGU/g DS, especially between 0.01-5 AGU/g DS, such as 0.1-2 AGU/g DS.

### Beta-Amylases

A beta-amylase (E.C 3.2.1.2) is the name traditionally given to exo-acting maltogenic amylases, which catalyze the hydrolysis of 1,4-alpha-glucosidic linkages in amylose, amylopectin and related glucose polymers. Maltose units are successively removed from the non-reducing chain ends in a step-wise manner until the molecule is degraded or, in the case of amylopectin, until a branch point is reached. The maltose released has the beta anomeric configuration, hence the name beta-amylase.

Beta-amylases have been isolated from various plants and microorganisms (Fogarty and Kelly, 1979, Progress in Industrial Microbiology 15: 112-115). These beta-amylases are characterized by having a temperature optimum in the range from 40° C. to 65° C. and a pH optimum in the range from 4.5 to 7. A commercially available beta-amylase from barley is NOVOZYM™ WBA from Novozymes A/S, Denmark, and SPEZYME™ BBA 1500 from Danisco, US Inc., USA.

### Maltogenic Amylases

The amylase may also be a maltogenic alpha-amylase. A “maltogenic alpha-amylase” (glucan 1,4-alpha-maltohydrolase, E.C. 3.2.1.133) is able to hydrolyze amylose and amylopectin to maltose in the alpha-configuration. A maltogenic amylase from Bacillus stearothermophilus strain NCIB 11837 is commercially available from Novozymes A/S. Maltogenic alpha-amylases are described in U.S. Pat. Nos. 4,598,048, 4,604,355 and 6,162,628, which are hereby incorporated by reference.

The maltogenic amylase may in a preferred embodiment be added in an amount of 0.05-5 mg total protein/gram DS or 0.05-5 MANU/g DS.

### Proteases

A protease may be added during saccharification, fermentation, simultaneous saccharification and fermentation. The protease may be any protease. In a preferred embodiment the protease is an acid protease of microbial origin, preferably of fungal or bacterial origin. An acid fungal protease is preferred, but also other proteases can be used.

Suitable proteases include microbial proteases, such as fungal and bacterial proteases. Preferred proteases are acidic proteases, i.e., proteases characterized by the ability to hydrolyze proteins under acidic conditions below pH 7.

The acid fungal protease may be derived from Aspergillus, Candida, Coriolus, Endothia, Enthomophtra, Irpex, Mucor, Penicillium, Rhizopus, Sclerotium, Torulopsis and Thermoascus.

In particular, the protease may be derived from Aspergillus aculeatus (WO 95/02044), Aspergillus awamori (Hayashida et al., 1977, Agric. Biol. Chem. 42(5), 927-933), Aspergillus niger (see, e.g., Koaze et al., 1964, Agr. Biol. Chem. Japan 28: 216), Aspergillus saitoi (see, e.g., Yoshida, 1954, J. Agr. Chem. Soc. Japan 28: 66), or Aspergillus oryzae, such as the pepA protease; acidic proteases from Mucor miehei or Mucor pusillus; the metallo protease from Thermoascus aurantiacus (AP025) disclosed as SEQ ID NO: 2 in WO 03/048353.

The protease may be a neutral or alkaline protease, such as a protease derived from a strain of Bacillus. A particular protease is derived from Bacillus amyloliquefaciens and has the sequence obtainable at Swissprot as Accession No. P06832. The proteases may have at least 90% sequence identity to amino acid sequence obtainable at Swissprot as Accession No. P06832 such as at least 92%, at least 95%, at least 96%, at least 97%, at least 98%, or particularly at least 99% identity.

The protease may have at least 90% sequence identity to the amino acid sequence disclosed as SEQ ID NO:1 in the WO 2003/048353 such as at 92%, at least 95%, at least 96%, at least 97%, at least 98%, or particularly at least 99% identity.

The protease may be a papain-like protease selected from the group consisting of proteases within E.C. 3.4.22.* (cysteine protease), such as EC 3.4.22.2 (papain), EC 3.4.22.6 (chymopapain), EC 3.4.22.7 (asclepain), EC 3.4.22.14 (actinidain), EC 3.4.22.15 (cathepsin L), EC 3.4.22.25 (glycyl endopeptidase) and EC 3.4.22.30 (caricain).

In an embodiment the protease is a protease preparation derived from a strain of Aspergillus, such as Aspergillus oryzae. In another embodiment the protease is derived from a strain of Rhizomucor, preferably Rhizomucor miehei. In another contemplated embodiment the protease is a protease preparation, preferably a mixture of a proteolytic preparation derived from a strain of Aspergillus, such as Aspergillus oryzae, and a protease derived from a strain of Rhizomucor, preferably Rhizomucor miehei.

Aspartic acid proteases are described in, for example, Handbook of Proteolytic Enzymes, Edited by A. J. Barrett, N. D. Rawlings and J. F. Woessner, Academic Press, San Diego, 1998, Chapter 270. Suitable examples of aspartic acid protease include, e.g., those disclosed in Berka et al., 1990, Gene 96: 313; Berka et al., 1993, Gene 125: 195-198; and Gomi et al., 1993, Biosci. Biotech. Biochem. 57: 1095-1100, which are hereby incorporated by reference.

Commercially available products include ALCALASE®, ESPERASE™, FLAVOURZYME™, PROMIX™, NEUTRASE®, RENNILASE®, NOVOZYM™ FM 2.0L, and iZyme BA (available from Novozymes A/S, Denmark) and GC106™ and SPEZYME™ FAN from Danisco US Inc., USA.

The protease may be present in an amount of 0.0001-1 mg enzyme protein per g DS, preferably 0.001 to 0.1 mg enzyme protein per g DS. Alternatively, the protease may be present in an amount of 0.0001 to 1 LAPU/g DS, preferably 0.001 to 0.1 LAPU/g DS and/or 0.0001 to 1 mAU-RH/g DS, preferably 0.001 to 0.1 mAU-RH/g DS.

### Compositions

In this aspect the invention relates to a composition comprising one or more deamidases.

In an embodiment the composition further comprises one or more other carbohydrases, such as alpha-amylases. In a preferred embodiment the alpha-amylase is an acid alpha-amylase or a fungal alpha-amylase, preferably an acid fungal alpha-amylase.

The composition may comprise one or more carbohydrate-source generating enzymes, such as especially glucoamylases, beta-amylases, maltogenic amylases, pullulanases, alpha-glucosidases, or a mixture thereof.

In another preferred embodiment the composition comprises one or more deamidases and further one or more fermenting organisms, such as yeast and/or bacteria. Examples of fermenting organisms can be found in the “Fermenting Organism” section above.

### Uses

In this aspect the invention relates to the use of deamidase in a fermentation process. In an embodiment a deamidase is used for improving the fermentation product yield. In another embodiment, a deamidase is used for increasing growth of the fermenting organism(s).

### Transgenic Plant Materials

In this aspect the invention relates to transgenic plant material transformed with one or more deamidase genes.

In one embodiment the invention relates to a transgenic plant, plant part, or plant cell which has been transformed with a polynucleotide sequence encoding a deamidase so as to express and produce the deamidase. The deamidase may be recovered from the plant or plant part, but in context of the present invention the plant or plant part containing the recombinant deamidase may be used in one or more of the methods or processes of the invention concerned and described above.

The transgenic plant can be dicotyledonous (a dicot) or monocotyledonous (a monocot). Examples of monocot plants are grasses, such as meadow grass (blue grass, Poa), forage grass such as Festuca, Lolium, temperate grass, such as Agrostis, and cereals, e.g., wheat, oats, rye, barley, rice, sorghum, and corn.

Examples of dicot plants are tobacco, legumes, such as lupins, potato, sugar beet, pea, bean and soybean, and cruciferous plants (family Brassicaceae), such as cauliflower, rape seed, and the closely related model organism Arabidopsis thaliana.

Examples of plant parts are stem, callus, leaves, root, fruits, seeds, and tubers as well as the individual tissues comprising these parts, e.g., epidermis, mesophyll, parenchyme, vascular tissues, meristems. Specific plant cell compartments, such as chloroplasts, apoplasts, mitochondria, vacuoles, peroxisomes and cytoplasm are also considered to be a plant part. Furthermore, any plant cell, whatever the tissue origin, is considered to be a plant part. Likewise, plant parts such as specific tissues and cells isolated to facilitate the utilisation of the invention are also considered plant parts, e.g., embryos, endosperms, aleurone and seeds coats.

Also included within the scope of the present invention are the progeny of such plants, plant parts, and plant cells.

The transgenic plant or plant cell expressing a deamidase may be constructed in accordance with methods well known in the art. In short, the plant or plant cell is constructed by incorporating one or more expression constructs encoding the deamidase into the plant host genome and propagating the resulting modified plant or plant cell into a transgenic plant or plant cell.

The expression construct is conveniently a nucleic acid construct which comprises a polynucleotide encoding a deamidase operably linked with appropriate regulatory sequences required for expression of the polynucleotide sequence in the plant or plant part of choice. Furthermore, the expression construct may comprise a selectable marker useful for identifying host cells into which the expression construct has been integrated and DNA sequences necessary for introduction of the construct into the plant in question (the latter depends on the DNA introduction method to be used).

The choice of regulatory sequences, such as promoter and terminator sequences and optionally signal or transit sequences, is determined, for example, on the basis of when, where, and how the enzyme is desired to be expressed. For instance, the expression of the gene encoding a deamidase may be constitutive or inducible, or may be developmental, stage or tissue specific, and the gene product may be targeted to a specific tissue or plant part such as seeds or leaves. Regulatory sequences are, for example, described by Tague et al., 1988, Plant Physiology 86: 506.

For constitutive expression, the ³⁵S-CaMV, the maize ubiquitin 1, and the rice actin 1 promoter may be used (Franck et al., 1980, Cell 21: 285-294, Christensen et al., 1992, Plant Mol. Biol. 18: 675-689; Zhang et al., 1991, Plant Cell 3: 1155-1165). Organ-specific promoters may be, for example, a promoter from storage sink tissues such as seeds, potato tubers, and fruits (Edwards & Coruzzi, 1990, Ann. Rev. Genet. 24: 275-303), or from metabolic sink tissues such as meristems (Ito et al., 1994, Plant Mol. Biol. 24: 863-878), a seed specific promoter such as the glutelin, prolamin, globulin, or albumin promoter from rice (Wu et al., 1998, Plant and Cell Physiology 39: 885-889), a Vicia faba promoter from the legumin B4 and the unknown seed protein gene from Vicia faba (Conrad et al., 1998, Journal of Plant Physiology 152: 708-711), a promoter from a seed oil body protein (Chen et al., 1998, Plant and Cell Physiology 39: 935-941), the storage protein napA promoter from Brassica napus, or any other seed specific promoter known in the art, e.g., as described in WO 91/14772. Furthermore, the promoter may be a leaf specific promoter such as the rbcs promoter from rice or tomato (Kyozuka et al., 1993, Plant Physiology 102: 991-1000, the chlorella virus adenine methyltransferase gene promoter (Mitra and Higgins, 1994, Plant Molecular Biology 26: 85-93), or the aldP gene promoter from rice (Kagaya et al., 1995, Molecular and General Genetics 248: 668-674), or a wound inducible promoter such as the potato pin2 promoter (Xu et al., 1993, Plant Molecular Biology 22: 573-588). Likewise, the promoter may inducible by abiotic treatments such as temperature, drought, or alterations in salinity or induced by exogenously applied substances that activate the promoter, e.g., ethanol, oestrogens, plant hormones such as ethylene, abscisic acid, and gibberellic acid, and heavy metals.

A promoter enhancer element may also be used to achieve higher expression of a deamidase in the plant. For instance, the promoter enhancer element may be an intron which is placed between the promoter and the polynucleotide sequence encoding a deamidase. For instance, Xu et al., 1993, supra, disclose the use of the first intron of the rice actin 1 gene to enhance expression.

The selectable marker gene and any other parts of the expression construct may be chosen from those available in the art.

The nucleic acid construct is incorporated into the plant genome according to conventional techniques known in the art, including Agrobacterium-mediated transformation, virus-mediated transformation, microinjection, particle bombardment, biolistic transformation, and electroporation (Gasser et al., 1990, Science 244: 1293; Potrykus, 1990, Bio/Technology 8: 535; and Shimamoto et al., 1989, Nature 338: 274).

Presently, Agrobacterium tumefaciens-mediated gene transfer is the method of choice for generating transgenic dicots (for a review, see Hooykas and Schilperoort, 1992, Plant Molecular Biology 19: 15-38) and can also be used for transforming monocots, although other transformation methods are often used for these plants. Presently, the method of choice for generating transgenic monocots is particle bombardment (microscopic gold or tungsten particles coated with the transforming DNA) of embryonic calli or developing embryos (Christou, 1992, Plant Journal 2: 275-281; Shimamoto, 1994, Current Opinion Biotechnology 5: 158-162; Vasil et al., 1992, Bio/Technology 10: 667-674). An alternative method for transformation of monocots is based on protoplast transformation as described by Omirulleh et al., 1993, Plant Molecular Biology 21: 415-428.

Following transformation, the transformants having incorporated the expression construct are selected and regenerated into whole plants according to methods well-known in the art. Often the transformation procedure is designed for the selective elimination of selection genes either during regeneration or in the following generations by using, for example, co-transformation with two separate T-DNA constructs or site specific excision of the selection gene by a specific recombinase.

A method for producing a deamidase in a plant comprises cultivating a transgenic plant or a plant cell comprising a polynucleotide encoding the deamidase under conditions conducive for production of the deamidase.

As mentioned above the transgenic plant material may be used in a method or process of the invention described above.

The transgenic plant is capable of expressing one or more deamidases in increased amounts compared to corresponding unmodified plant material.

### Modified Fermenting Organisms

In this aspect the invention relates to a modified fermenting organism transformed with a polynucleotide encoding a deamidase, wherein the fermenting organism is capable of expressing the deamidase at fermentation conditions.

In a preferred embodiment the fermentation conditions are as defined according to the invention. In a preferred embodiment the fermenting organism is a microbial organism, such as yeast or filamentous fungus, or a bacterium. Examples of other fermenting organisms can be found in the“Fermenting Organisms” section.

A fermenting organism may be transformed with a deamidase encoding gene using techniques well know in the art.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure, including definitions will be controlling.

Various references are cited herein, the disclosures of which are incorporated by reference in their entireties.

### Materials & Methods

### Methods:

### Identity

The relatedness between two amino acid sequences or between two polynucleotide sequences is described by the parameter “identity”.

For purposes of the present invention, the degree of identity between two amino acid sequences is determined by the Clustal method (Higgins, 1989, CABIOS 5: 151-153) using the LASERGENE™ MEGALIGN™ software (DNASTAR, Inc., Madison, Wis.) with an identity table and the following multiple alignment parameters: Gap penalty of 10 and gap length penalty of 10. Pairwise alignment parameters are Ktuple=1, gap penalty=3, windows=5, and diagonals=5.

For purposes of the present invention, the degree of identity between two polynucleotide sequences is determined by the Wilbur-Lipman method (Wilbur and Lipman, 1983, Proceedings of the National Academy of Science USA 80: 726-730) using the LASERGENE™ MEGALIGN™ software (DNASTAR, Inc., Madison, Wis.) with an identity table and the following multiple alignment parameters: Gap penalty of 10 and gap length penalty of 10. Pairwise alignment parameters are Ktuple=3, gap penalty=3, and windows=20.

### Deamidase Activity Assay:

Deamidase activity can be measured by following the formation of ammonia using the Ammonia kit (Modified Fujii-Okuda method) from Wako, cat. #277-14401.

### Assay Principle:

1. Ammonia is developed by the deamidating action of the enzyme.
2. Ammonia reacts with phenol to form dioxyphenylamine under alkaline conditions. The reaction is catalyzed by sodium penta-cyanonitrosylferrate(III) (sodium nitroprusside). “Color Reagent solution A” contains phenol and sodium nitroprusside. “Color Reagent Solution B” provides alkaline reaction conditions.
3. The intermediate is then oxidized by addition of sodium hypochlorite (“Color Reagent Solution C”) to form indophenol blue. This compound absorbs visible light at 630 nm.

### Assay Procedure:

1. Transfer 10 microliters of enzyme solution in an appropriate buffer (in terms of pH and not consuming the formed ammonia) to a 1.5 mL Eppendorf tube.
2. Mix with 240 microliters of substrate solution (8-10 mM Z-Gln-Gly (Sigma C-6154)).
3. Incubate with shaking (750 rpm) @ 37° C. for 15 minutes.
4. Transfer the samples (reaction mixture) on ice.
5. Mix 200 microliters of reaction mixture with 800 microliters of “Deproteinizing Reagent Solution” from the Ammonia kit in a 1.5 ml Eppendorf tube. Vortex.
6. Centrifugation @ 16100×G, 10° C., for 5 minutes.
7. Transfer 400 microliters of supernatant to a new 1.5 ml Eppendorf tube and add 400 microliters of “Color Reagent Solution A” from the Ammonia kit. Vortex.
8. Add 200 microliters of “Color Reagent Solution B” from the Ammonia kit. Vortex.
9. Add 400 microliters of “Color Reagent Solution C” from the Ammonia kit. Vortex.
10. Color development: Incubate the samples with shaking (750 rpm) @ 37° C. for 20 minutes.
11. Transfer the samples on ice and allow to cool for 10 min.
12. Read absorbance @ 630 nm within 1 hour of finishing the assay.

### Standard Curve:

| No. | St. Solution | Diluent for St. Sol.^{*} | Conc. of NH₃—N |
|---|---|---|---|
| 1 | 100 microliters | 300 microliters | 100 micrograms/dL |
| 2 | 200 microliters | 200 microliters | 200 micrograms/dL |
| 3 | 300 microliters | 100 microliters | 300 micrograms/dL |
| 4 | 400 microliters | 0 microliters | 400 micrograms/dL |

^{*}Part of the Ammonia kit from Wako.

1. Mix 200 microliters of diluted standard solution (according to the scheme above) with 800 microliters of “Deproteinizing Reagent Solution” from the Ammonia kit. Vortex.
2. Perform assay as described from point 6 above.

### Reagent Blank:

1. Mix 200 microliters of buffer with 800 microliters of “Deproteinizing Reagent Solution” from the Ammonia kit. Vortex.
2. Perform assay as described from point 6 above.

### Calculation:

Plot absorbance of standard solutions (minus blank) along the ordinate against the ammonia concentration along the abscissa.

Formed ammonia during reaction:

Ammonium-nitrogen (micrograms/dL)=((A_{S}-A_{B})/(A₄₀₀-A_{B}))*400

Aₛ: Absorbance of sample

A_{B}: Absorbance of blank

A₄₀₀: Absorbance of standard solution no. 4 in the table above

The amount of enzyme which releases 1 micromol of ammonia per minute under the above reaction conditions is defined as one unit and calculated based on the following formula:

U/ml=0.39*((A_{S}−A_{B})/(A₄₀₀-A_{B}))

### Glucoamylase Activity

Glucoamylase activity may be measured in Glucoamylase Units (AGU).

### Glucoamylase Activity (AGU)

The Novo Glucoamylase Unit (AGU) is defined as the amount of enzyme, which hydrolyzes 1 micromole maltose per minute under the standard conditions 37° C., pH 4.3, substrate: maltose 23.2 mM, buffer: acetate 0.1 M, reaction time 5 minutes.

An autoanalyzer system may be used. Mutarotase is added to the glucose dehydrogenase reagent so that any alpha-D-glucose present is turned into beta-D-glucose. Glucose dehydrogenase reacts specifically with beta-D-glucose in the reaction mentioned above, forming NADH which is determined using a photometer at 340 nm as a measure of the original glucose concentration.

| AMG incubation: | |
|---|---|
| Substrate: | maltose 23.2 mM |
| Buffer: | acetate 0.1 M |
| pH: | 4.30 ± 0.05 |
| Incubation temperature: | 37° C. ± 1 |
| Reaction time: | 5 minutes |
| Enzyme working range: | 0.5-4.0 AGU/mL |

| Color reaction: | |
|---|---|
| GlucDH: | 430 U/L |
| Mutarotase: | 9 U/L |
| NAD: | 0.21 mM |
| Buffer: | phosphate 0.12 M; |
| | 0.15 M NaCl |
| pH: | 7.60 ± 0.05 |
| Incubation temperature: | 37° C. ± 1 |
| Reaction time: | 5 minutes |
| Wavelength: | 340 nm |

### Alpha-Amylase Activity (KNU)

The alpha-amylase activity may be determined using potato starch as substrate. This method is based on the break-down of modified potato starch by the enzyme, and the reaction is followed by mixing samples of the starch/enzyme solution with an iodine solution. Initially, a blackish-blue color is formed, but during the break-down of the starch the blue color gets weaker and gradually turns into a reddish-brown, which is compared to a colored glass standard.

One Kilo Novo alpha amylase Unit (KNU) is defined as the amount of enzyme which, under standard conditions (i.e., at 37° C.+/−0.05; 0.0003 M Ca²⁺; and pH 5.6) dextrinizes 5260 mg starch dry substance Merck Amylum solubile.

### Acid Alpha-Amylase Activity

When used according to the present invention the activity of an acid alpha-amylase may be measured in AFAU (Acid Fungal Alpha-amylase Units) or FAU-F.

### Acid Alpha-Amylase Activity (AFAU)

Acid alpha-amylase activity may be measured in AFAU (Acid Fungal Alpha-amylase Units), which are determined relative to an enzyme standard. 1 AFAU is defined as the amount of enzyme which degrades 5.260 mg starch dry matter per hour under the below mentioned standard conditions.

Acid alpha-amylase, an endo-alpha-amylase (1,4-alpha-D-glucan-glucanohydrolase, E.C. 3.2.1.1) hydrolyzes alpha-1,4-glucosidic bonds in the inner regions of the starch molecule to form dextrins and oligosaccharides with different chain lengths. The intensity of color formed with iodine is directly proportional to the concentration of starch. Amylase activity is determined using reverse colorimetry as a reduction in the concentration of starch under the specified analytical conditions.

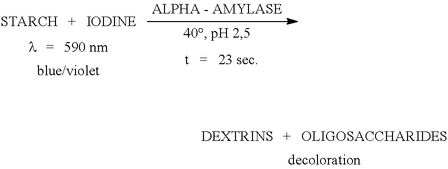

| Standard conditions/reaction conditions: | |
|---|---|
| Substrate: | Soluble starch, approx. |
| | 0.17 g/L |
| Buffer: | Citrate, approx. 0.03 M |
| Iodine (I₂): | 0.03 g/L |
| CaCl₂: | 1.85 mM |
| pH: | 2.50 ± 0.05 |
| Incubation temperature: | 40° C. |
| Reaction time: | 23 seconds |
| Wavelength: | 590 nm |
| Enzyme concentration: | 0.025 AFAU/mL |
| Enzyme working range: | 0.01-0.04 AFAU/mL |

### Determination of FAU-F

FAU-F Fungal Alpha-Amylase Units (Fungamyl) is measured relative to an enzyme standard of a declared strength.

| Reaction conditions | |
|---|---|
| Temperature | 37° C. |
| pH | 7.15 |
| Wavelength | 405 nm |
| Reaction time | 5 min |
| Measuring time | 2 min |

### Protease Assay Method—AU(RH)

The proteolytic activity may be determined with denatured hemoglobin as substrate. In the Anson-Hemoglobin method for the determination of proteolytic activity denatured hemoglobin is digested, and the undigested hemoglobin is precipitated with trichloroacetic acid (TCA). The amount of TCA soluble product is determined with phenol reagent, which gives a blue color with tyrosine and tryptophan.

One Anson Unit (AU-RH) is defined as the amount of enzyme which under standard conditions (i.e., 25° C., pH 5.5 and 10 minutes reaction time) digests hemoglobin at an initial rate such that there is liberated per minute an amount of TCA soluble product which gives the same color with phenol reagent as one milliequivalent of tyrosine.

The AU(RH) method is described in EAL-SM-0350 and is available from Novozymes A/S Denmark on request.

### Protease Assay Method (LAPU)

1 Leucine Amino Peptidase Unit (LAPU) is the amount of enzyme which decomposes 1 microM substrate per minute at the following conditions: 26 mM of L-leucine-p-nitroanilide as substrate, 0.1 M Tris buffer (pH 8.0), 37° C., 10 minutes reaction time.

LAPU is described in EB-SM-0298.02/01 available from Novozymes A/S Denmark on request.

### Determination of Maltogenic Amylase Activity (MANU)

One MANU (Maltogenic Amylase Novo Unit) may be defined as the amount of enzyme required to release one micromole of maltose per minute at a concentration of 10 mg of maltotriose (Sigma M 8378) substrate per ml of 0.1 M citrate buffer, pH 5.0 at 37° C. for 30 minutes.

### Materials:

Alpha-Amylase A (AA): Hybrid alpha-amylase consisting of Rhizomucor pusillus alpha-amylase with Aspergillus niger glucoamylase linker and SBD disclosed as V039 in Table 5 in WO 2006/069290 (Novozymes A/S).
Deamidase: Deamidase derived from Chryseobacterium gleum disclosed in SEQ ID NO: 6 in U.S. Pat. No. 6,251,651.
Glucoamylase (GA): Glucoamylase derived from Trametes cingulata disclosed in SEQ ID NO: 2 in WO 2006/069289 and available from Novozymes A/S.
Yeast: RED START™ available from Red Star/Lesaffre, USA.

### EXAMPLES

### Example 1

### Effect of Deamidase Towards Alpha-Amylase (AA) and Glucoamylase (GA) in a One-Step Simultaneous Saccharification and Fermentation (SSF) Process

All treatments were evaluated via mini-scale fermentations. Four hundred and ten g of ground yellow dent corn (with an average particle size around 0.5 mm) was added to 590 g tap water. This mixture was supplemented with 3.0 ml of a 1 g/L penicillin stock solution and 1 g of urea. The pH of this slurry was adjusted to 4.5 with 40% H₂SO₄. Dry solid (DS) level was determined to be 35 wt. %. Approximately 5 g of this slurry was added to 20 ml vials. Each vial was dosed with alpha-amylase, glucoamylase, and deamidase at the dosages shown in Table 1 followed by the addition of 200 microliters yeast propagate/5 g slurry. Actual enzyme dosages were based on the exact weight of corn slurry in each vial.

TABLE 1

| | | AA dose | GA dose | Deamidase dose |
|---|---|---|---|---|
| | | (FAU-F/g | (AGU/g | (micro- |
| | Treatments | DS) | DS) | grams/g DS) |
| 1 | AA + GA (V) | 0.0475 | 0.5 | 0 |
| 2 | AA + GA + Deamidase (W) | 0.0475 | 0.5 | 20 |
| 3 | AA + GA + Deamidase (X) | 0.0475 | 0.5 | 40 |
| 4 | AA + GA + Deamidase (Y) | 0.0475 | 0.5 | 80 |
| 5 | AA + GA + Deamidase (Z) | 0.0475 | 0.5 | 100 |

The vials were incubated at 32° C. without agitation, and mixed once per day. Nine replicate fermentations of each treatment were run. Three replicates were run for 24 hours, 48 hours and 70 hours time point analysis. Vials were vortexed at 24, 48 and 70 hours and analyzed by HPLC. Samples for HPLC analysis were prepared by adding 50 microliters of 40% H₂SO₄, centrifuging, and filtering through a 0.45 micrometer filter. The samples were stored at 4° C. until analysis. An Agilent™ 1100 HPLC system (Agilent Technologies, Santa Clara, Calif., USA) equipped with a 7.8×300 mm AMINEX® HPX-87H column (Bio-Rad Laboratories, Inc., Hercules, Calif., USA) and coupled with a refractive index (R1) detector was used to determine the ethanol concentration.

The results are provided in Table 2.

TABLE 2
Ethanol yield (g/L) with time and deamidase at different concentrations

| | Deamidase | | | | |
|---|---|---|---|---|---|
| Time | V | W | X | Y | Z |
| 24 hours | 100.66 +/− 1.94 | 99.45 +/− 1.47 | 99.76 +/− 1.45 | 99.73 +/− 0.39 | 99.51 +/− 0.26 |
| 48 hours | 139.59 +/− 0.54 | 141.41 +/− 0.14 | 141.75 +/− 0.39 | 141.29 +/− 0.89 | 141.72 +/− 0.18 |
| 70 hours | 149.09 +/− 0.50 | 151.59 +/− 0.75 | 151.22 +/− 0.18 | 152.11 +/− 0.44 | 152.55 +/− 0.22 |

The results show a commercially significant increase in ethanol production and higher fermentation kinetics after 48 and 70 hours.

The present invention is further described by the following numbered paragraphs:

[1] A process of producing a fermentation product, comprising:

(a) converting a starch-containing material to dextrins with an alpha-amylase;

(b) saccharifying the dextrins to a sugar with a glucoamylase;

(c) adding a deamidase; and

(d) fermenting the sugar using a fermenting organism.

[2] The process of paragraph [1], wherein the starch-containing material is converted to dextrins by liquefaction.
[3] The process of paragraph [2], wherein the liquefaction comprises jet-cooking at a temperature between 95-140° C.
[4] The process of paragraph [2] or [3], further comprising pre-saccharification of typically 40-90 minutes at a temperature between 30-65° C.
[5] The process of any of paragraphs [2]-[4], wherein the saccharification is carried out at a temperature in the range of 20-75° C.
[6] The process of any of paragraphs [2]-[5], further comprising a pre-saccharification prior to saccharification.
[7] The process of any of paragraphs [2]-[6], wherein the deamidase is added during the conversion of the starch-containing materials to dextrins.
[8]. The process of any of paragraphs [2]-[7], wherein the deamidase is added during the saccharification of the dextrins to a sugar.
[9] The process of any of paragraphs [2]-[8], wherein the deamidase is added during pre-saccharification.
[10] The process of any of paragraphs [2]-[9], wherein the deamidase is added during the fermentation.
[11] The process of any of paragraphs [2]-[10], wherein the saccharification and fermentation are performed simultaneously.
[12] The process of paragraph [11], wherein the saccharification and fermentation are carried out at a temperature in the range of 20° C. to 40° C.
[13] The process of paragraph [1], wherein the starch-containing material is converted to dextrins and the dextrins are saccharified to a sugar by treating the starch-containing material with an alpha-amylase and glucoamylase below the initial gelatinization temperature of the starch-containing material.
[14] The process of paragraph [13], wherein the conversion of the starch-containing material to dextrins, the saccharification of the dextrins to a sugar, and the fermentation of the sugar are carried out in a single step.
[15] The process of paragraph [13] or [14], wherein the alpha-amylase, glucoamylase, fermentation organism, and deamidase are added simultaneously or sequentially.
[16] The process of any of paragraphs [13]-[15], which is carried out at a temperature between 25° C. and 40° C.
[17] The process of any of paragraphs [1]-[16], wherein the starch-containing material is selected from the group consisting of barley, beans, cassaya, cereals, corn, milo, peas, potatoes, rice, rye, sago, sorghum, sweet potatoes, tapioca, wheat, and whole grains, or any mixture thereof.
[18] The process of any of paragraphs [1]-[17], wherein the fermentation product is selected from the group consisting of alcohols (e.g., ethanol, methanol, butanol, 1,3-propanediol); organic acids (e.g., citric acid, acetic acid, itaconic acid, lactic acid, gluconic acid, gluconate, lactic acid, succinic acid, 2,5-diketo-D-gluconic acid); ketones (e.g., acetone); amino acids (e.g., glutamic acid); gases (e.g., H₂ and CO₂), and more complex compounds, including, for example, antibiotics (e.g., penicillin and tetracycline); enzymes; vitamins (e.g., riboflavin, B₁₂, beta-carotene); and hormones.
[19] The process of paragraph [18], wherein the fermentation product is ethanol.
[20] The process of any of paragraphs [1]-[19], further comprising recovering the fermentation product.
[21] The process of paragraph [20], wherein the fermentation product is recovered by distillation.
[22] Use of a deamidase in a fermentation process.
[23] The use of paragraph [22], wherein the fermentation process is a process for producing ethanol.
[24] A modified fermenting organism transformed with a polynucleotide encoding a deamidase, wherein the fermenting organism is capable of expressing the deamidase at fermentation conditions.
[25] A composition comprising a deamidase, a glucoamylase and an alpha-amylase.
[26] The composition of paragraph 25, further comprising a pullulanase.

1. A process of producing a fermentation product, comprising:
(a) converting a starch-containing material to dextrins with an alpha-amylase;
(b) saccharifying the dextrins to a sugar with a glucoamylase;
(c) adding a deamidase; and
(d) fermenting the sugar using a fermenting organism.

2. The process of claim 1, wherein the deamidase is added during the conversion of the starch-containing materials to dextrins.

3. The process of claim 2, wherein the deamidase is added during the saccharification of the dextrins to a sugar.

4. The process of claim 2, wherein the deamidase is added during the fermentation.

5. The process of claim 2, wherein the saccharification and fermentation are performed simultaneously.

6. The process of claim 1, wherein the starch-containing material is converted to dextrins and the dextrins are saccharified to a sugar by treating the starch-containing material with an alpha-amylase and glucoamylase below the initial gelatinization temperature of the starch-containing material.

7. The process of claim 6, wherein the conversion of the starch-containing material to dextrins, the saccharification of the dextrins to a sugar, and the fermentation of the sugar are carried out in a single step.

8. The process of claim 6, wherein the alpha-amylase, glucoamylase, fermentation organism, and deamidase are added simultaneously or sequentially.

9. The process of claim 6, which is carried out at a temperature between 25° C. and 40° C.

10. The process of claim 1, wherein the fermentation product is selected from the group consisting of alcohols (e.g., ethanol, methanol, butanol, 1,3-propanediol); organic acids (e.g., citric acid, acetic acid, itaconic acid, lactic acid, gluconic acid, gluconate, lactic acid, succinic acid, 2,5-diketo-D-gluconic acid); ketones (e.g., acetone); amino acids (e.g., glutamic acid); gases (e.g., H₂ and CO₂), and more complex compounds, including, for example, antibiotics (e.g., penicillin and tetracycline); enzymes; vitamins (e.g., riboflavin, B₁₂, beta-carotene); and hormones.

11. The process of claim 10, wherein the fermentation product is ethanol.

12. The process of claim 1, further comprising recovering the fermentation product.

13. (canceled)

14. A modified fermenting organism transformed with a polynucleotide encoding a deamidase, wherein the fermenting organism is capable of expressing the deamidase at fermentation conditions.

15. A composition comprising a deamidase, a glucoamylase and an alpha-amylase.
